(19) United States
(12) Patent Application Publication
(10) Pub. No.: US 2018/0279880 A1
(43) Pub. Date: Oct. 4, 2018

(54) SYSTEM AND METHOD FOR ENHANCED PATIENT MONITORING AND CARE

(71) Applicant: Anthony J. Bacchi, West Hurley, NY (US)

(72) Inventor: Anthony J. Bacchi, West Hurley, NY (US)

(21) Appl. No.: 15/827,282

(22) Filed: Nov. 30, 2017

Related U.S. Application Data
(60) Provisional application No. 62/428,355, filed on Nov. 30, 2016.

(51) Int. Cl. A61B 5/00; G16H 40/67; G16H 10/60; A61B 5/145

(52) U.S. Cl. CPC A61B 5/0022; G16H 40/67; G16H 10/60; A61B 5/6833; A61B 5/4848; A61B 5/4842; A61B 5/14532

(57) ABSTRACT

A clinical healthcare system and method comprising specialized hardware and software components is described in which data collected from a plurality of patient-monitoring devices is provided to a remote care management unit for real-time monitoring and management. A portable computing device such as a tablet computer or smart television is configured with a patient-facing that provides information on treatment and care and facilitates communication with healthcare providers. Continuous patient monitoring by healthcare professionals reduces the rate of relapse or readmission.

### PRIORITY CLAIM

This application claims priority to U.S. Patent Application No. 62/428,355, filed Nov. 30, 2016 and titled, “SYSTEM AND METHOD FOR ENHANCED PATIENT MONITORING AND CARE,” the contents of which is incorporated by reference in its entirety.

### BACKGROUND

Patient relapse after treatment continues to be a significant problem in the healthcare industry, particularly among elderly populations. A patient discharged from a hospital after a heart attack, stroke, hypertensive episode, or other medical issue may be provided with a treatment plan that can involve diet, medication, follow-up appointments, and monitoring of symptoms and vital signs. Many patients fail to follow these treatment plans and it has been noticed that there is an acute relapse and readmission rate amongst certain populations, particularly the elderly.

Relapse and readmission is a multi-billion-dollar issue, spread across healthcare providers, insurance companies, state and federal governments, patients, and their families. An economic and social cost is imposed when patients are readmitted to the hospital, and removed from their homes. In acute cases, the patient may require a stay in a nursing home with minimal autonomy.

It has been found that by providing a system and method for in-home patient monitoring that utilizes specialized sensor and computing devices that are continuously monitored by healthcare professionals, the rate of relapse or readmission can be substantially reduced.

### SUMMARY OF THE INVENTION

In embodiments of the invention, a method is described for evaluating patient health and reducing patient relapse after treatment that comprises the steps of: (1) establishing a communication channel with at least one patient monitoring device that has been configured to measure a physiological characteristic of a patient; (2) establishing a second communication channel with a remote care management unit; (3) receiving a first patient data object describing the physiological characteristic of a patient, evaluating the first patient data object against predetermined criteria, and causing an alert condition when the first patient data object is outside the predetermined criteria; and (4) receiving the alert condition at the remote care management unit and facilitating a communication link between the portable communication device and the remote care management unit such that the need for further intervention can be determined.

In alternate embodiments of the invention, a non-transitory computer readable storage medium is provided and stores one or more sequences of instructions, that when executed by one or more processors, to cause the one or more processors to perform a set of operations comprising: (1) establishing a communication channel with at least one patient monitoring device that has been configured to measure a physiological characteristic of a patient; (2) establishing a second communication channel with a remote care management unit; (3) receiving a first patient data object describing the physiological characteristic of a patient, evaluating the first patient data object against predetermined criteria, and causing an alert condition when the first patient data object is outside the predetermined criteria; and (4) receiving the alert condition at the remote care management unit and facilitating a communication link between the portable communication device and the remote care management unit such that the need for further intervention can be determined.

In embodiments of the invention the portable computing device may be one of: a smartphone computing device, a tablet computing device, and a laptop computing device, or one of: a smart television device or a streaming device. In embodiments, the patient monitoring device may be a telemetry device used to monitor heart rate and rhythm, and may be connected to the patient using a disposable patch. In embodiments, the hemodynamic data may include electrocardiograms data from a monitoring device having at least six leads. In embodiments, the remote care management unit may receive hemodynamic data in real time over the second communication channel, and the remote care monitoring unit may be a central facility staffed by healthcare professionals to monitor patient wellness. In embodiments, the remote care monitoring unit may comprise at least one cognitive computing device configured to analyze all incoming patient data objects data to identify potential critical care situations. In further embodiments, the portable computing device may be configured to provide interactive access to at least one of: a patient medication portal, a diet and nutrition portal, a physical therapy portal, an illness portal, a vital signs portal, and an appointments portal.

### BRIEF DESCRIPTION OF THE DRAWINGS

The features and advantages of the present disclosure will be more fully understood with reference to the following detailed description when taken in conjunction with the accompanying figures, wherein:

FIG. 1 shows an exemplary set of sensors and data sources utilized in embodiments of the invention.

FIG. 2 shows a sample home screen of a portable computing device used with embodiments of the present invention.

FIGS. 3a and 3b show, respectively, a monitoring station view of a patient's blood pressure trend over time and a view of a patient's bloodwork and trends over time.

FIG. 4 shows a medication portal provided on a patient device used with embodiments of the present invention.

FIG. 5 shows a diet and nutrition portal provided on a patient device used with embodiments of the present invention.

FIG. 6 shows an exercise and physical therapy portal provided on a patient device used with embodiments of the present invention.

FIG. 7 shows an illness portal provided on a patient device used with embodiments of the present invention.

FIG. 8 shows a vital signs portal provided on a patient device used with embodiments of the present invention.

FIG. 9 shows an appointments and scheduling portal provided on a patient device used with embodiments of the present invention.

FIG. 10 is a block diagram depicting a holistic overview of an exemplary embodiment of the present invention.

### DETAILED DESCRIPTION

A clinical healthcare system and method comprising specialized hardware and software components is described in which the gap between the patient at home and caregiver is bridged and real-time monitoring of patient health is enabled. Beyond monitoring and data collection, the present invention can educate the user and caregiver and/or family member on when medication must be taken, what exercises should be performed, what diet should be followed, pertinent health tips, appointment times, and when there is a change in conditions, among many others.

An exemplary system may comprise three discrete components: (1) sensors for monitoring patient health and well-being; (2) a central monitoring unit that aggregates sensor data and watches for critical or acute conditions; and (3) a portable computing device that enables two-way communication between the patient, the central monitoring unit, and healthcare providers. The system and method of the present invention thus allows patients to remain home while being monitored by clinical professionals in real time with hemodynamic data.

In embodiments, a plurality of sensors may be provided for monitoring patient vital signs, and may include monitoring of: cardiac rhythm (6-lead), blood pressure, oxygen level, respiratory rate, cardiac output, stroke volume, systemic vascular resistance, and cardiac index, among others.

Data from these sensors is gathered continuously or near-continuously to provide a detailed picture of the health of the patient. Where the ability to monitor patient data over time is enabled, a developing condition may be identified prior to the onset of a medical emergency. For example, a steady but modest increase in blood pressure over a period of days may present a different than a single-day increase. Appropriate treatment options may then be considered.

It will be appreciated by those of skill in the art that any combination of sensors may be utilized with the primary consideration being the ability to collect sufficient patient data to evaluate the patient's condition and identify and incipient health issues.

In embodiments, patient data gathered by an array of sensors may be stored locally on a computing device in the patient's home. Alternatively, patient data may be collected and transmitted to a central facility in real time, as described below. Sensors should thus, preferably, be configured to communicate with a mobile computing device—such as by Bluetooth, Wi-Fi, cellular, or even USB—to aggregate and display data for the patient, and also to transmit the data to a central monitoring unit for analysis. Configuration of the patient-side elements of the present invention are preferably easy to install and set up and require limited involvement or maintenance on the part of the patient.

In embodiments, data collected from the patient may be gathered and transmitted by any of the sensors comprising the sensor array, either with or without the involvement of the portable computing device.

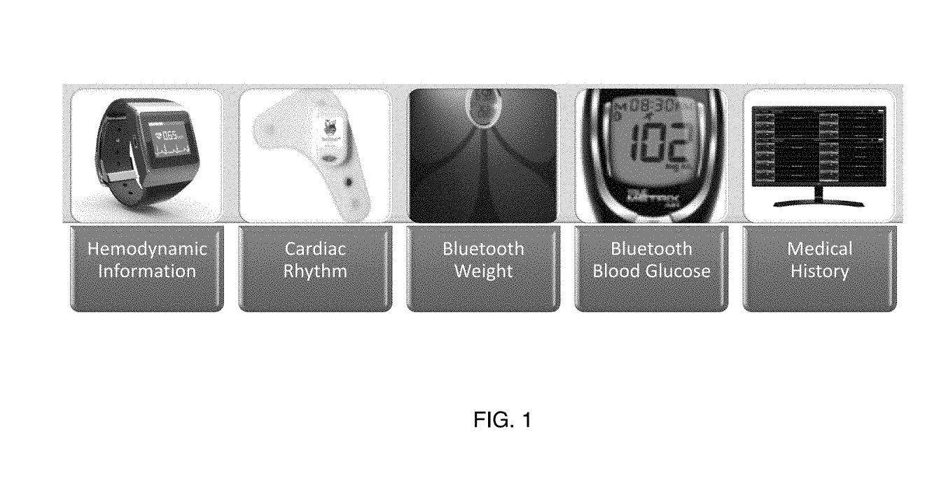

FIG. 1 shows an array of exemplary set of sensors and data sources that may be used with embodiments of the invention. Hemodynamic parameters may be monitored by devices such as a smartwatch to provide data on blood pressure, cardiac output, and the like. Cardiac rhythm may be monitored via a sensor responsive to electrical activity in the heart to identify conditions such as atrial fibrillation. An electronic scale may be connected to the system via Bluetooth or other wireless protocol to provide information on a patient's weight over time. Similarly, patients using a blood glucose monitor to monitor the level of glucose in the bloodstream may link the monitor to the system to provide blood glucose information over time.

In embodiments, a telemetry device may be used to monitor heart rate and rhythm. Electrodes attached to the patient may then transmit data directly to a central monitoring unit, or to an associated portable computing device. Telemetry devices may be configured to connect to a portable communication device, or to connect to a cellular, Wi-Fi, or other networks to transmit the data directly. Telemetry devices may attach via a disposable patch that includes the electrodes and is wirelessly connected to the system.

In embodiments, patient monitoring hardware will be remotely connected to a central monitoring unit (CMU), which is a central facility staffed by healthcare professionals to monitor patient wellness.

As will be discussed below, a portable computing device may enable a patient to communicate directly with the healthcare professionals in the CMU concerning their condition. In embodiments, a portable computing device may take the form of a tablet computer, smartphone, or smart television, and be provided to a patient preconfigured to interact with the other elements of the system. In embodiments, elements of the system may be integrated on portable computing devices such as digital media players and microconsoles, including the Apple TV, Roku, Google Chromecast, and Amazon Fire TV Stick devices, among others.

A portable computing device for use with the present system may be preconfigured to facilitate data exchange between patient and caregivers, facilitate communication between patient and caregiver, and provide information to patient.

It should be noted that references herein to a “personal computing device” are not limited to smartphones, tablet computers, laptop computers, and smart televisions, and can encompass any electronic device—whether for communication, entertainment, or otherwise—that can be delivered to the home and configured to provide an interactive communication experience with a remote CMU.

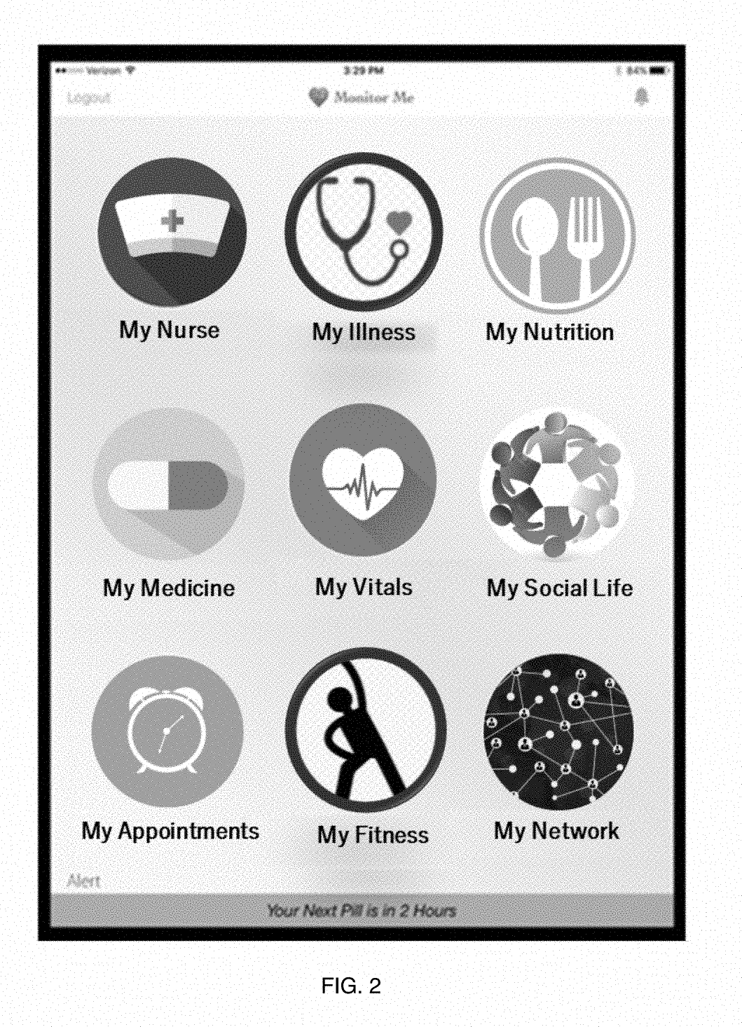

A sample home screen for the portable computing device is shown in FIG. 2. As shown, the sample home screen may have multiple icons (in this example, nine) laid out in an easy-to-read and easy-to-select format. Icons are provided for a variety of features that may be loosely grouped as reminders, wellness, and information.

A nurse function may be provided to enable the patient to call a nurse or doctor using the videoconferencing features of the portable computing device or television, and allows patients to speak directly with a nurse in real-time by the push of a button.

During such a videoconference, the nurse may log additional information and also conference the doctor to discuss any problems the patient is having. The nurse is in a monitoring station and can see the patient's medical history and hemodynamic monitoring in real-time.

In embodiments, the nurse function may function as a pager function that sends a message to the nurse requesting a call by traditional means. If a higher level of care is required the nurse can immediately connect the patient to a doctor using this technology.

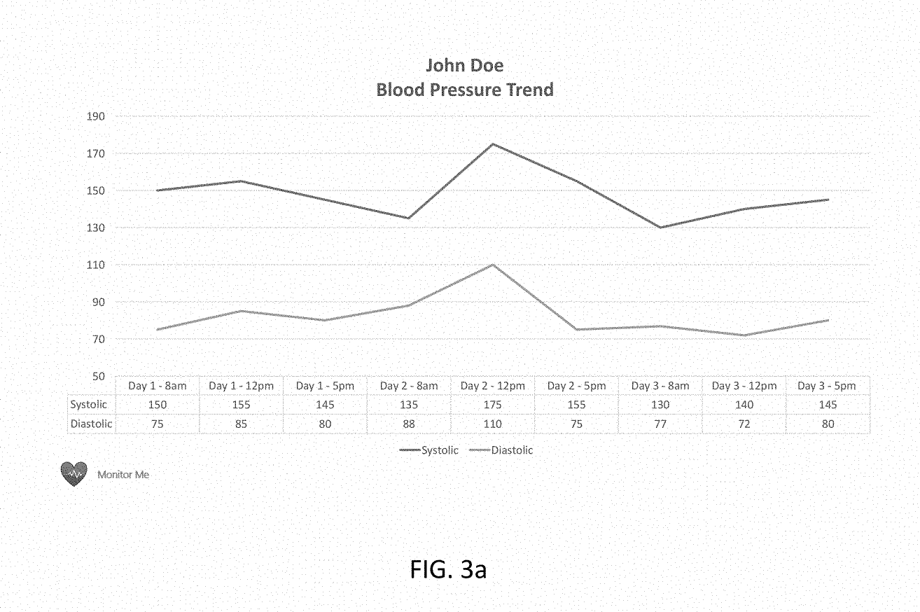

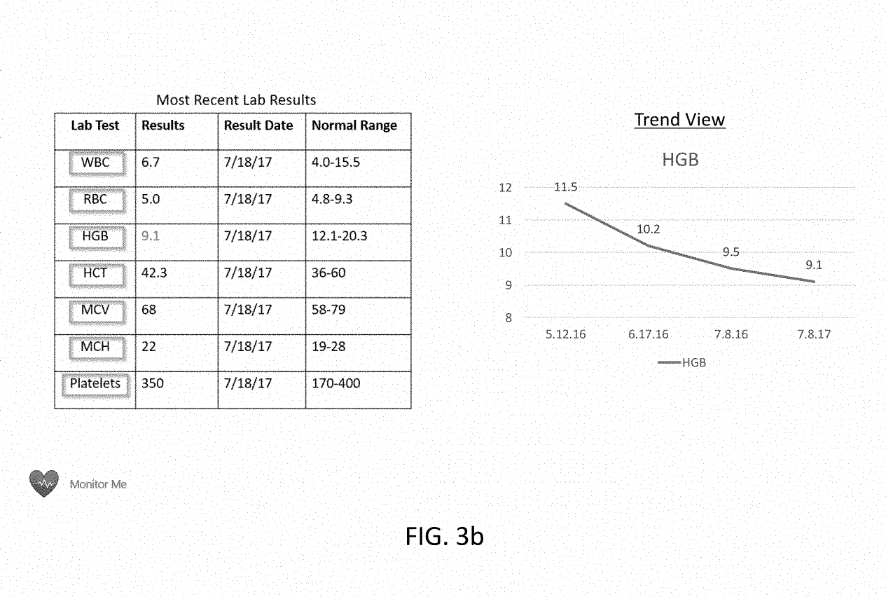

A monitoring station used with embodiments of the invention may show patient information during a one-on-one interaction with a patient, and also for multiple patients simultaneously. FIGS. 3a and 3b show, respectively, a monitoring station view of a patient's blood pressure trend over time and a view of a patient's bloodwork and trends over time.

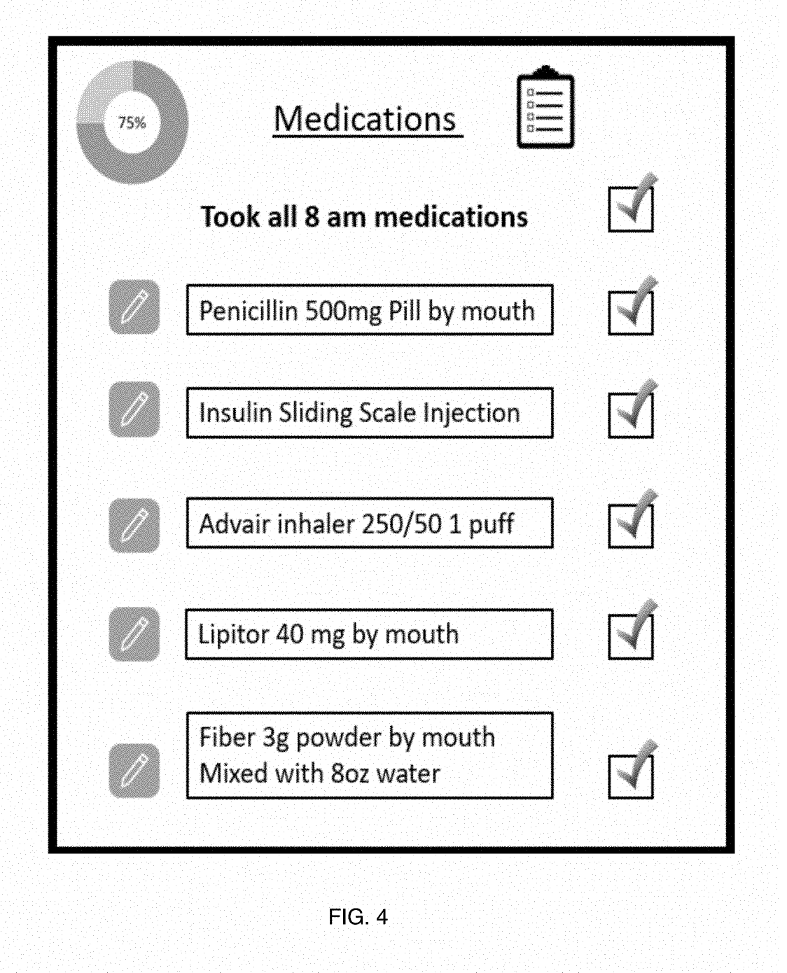

A My Medication interface, shown in FIG. 4, may provide the patient with critical information concerning medications that must be taken, provide alters to take medication, and provide monitoring of a patient's adherence to the prescription. This module may also provide an overview of all medications taken by the patient and specific instructions or warnings concerning those medications.

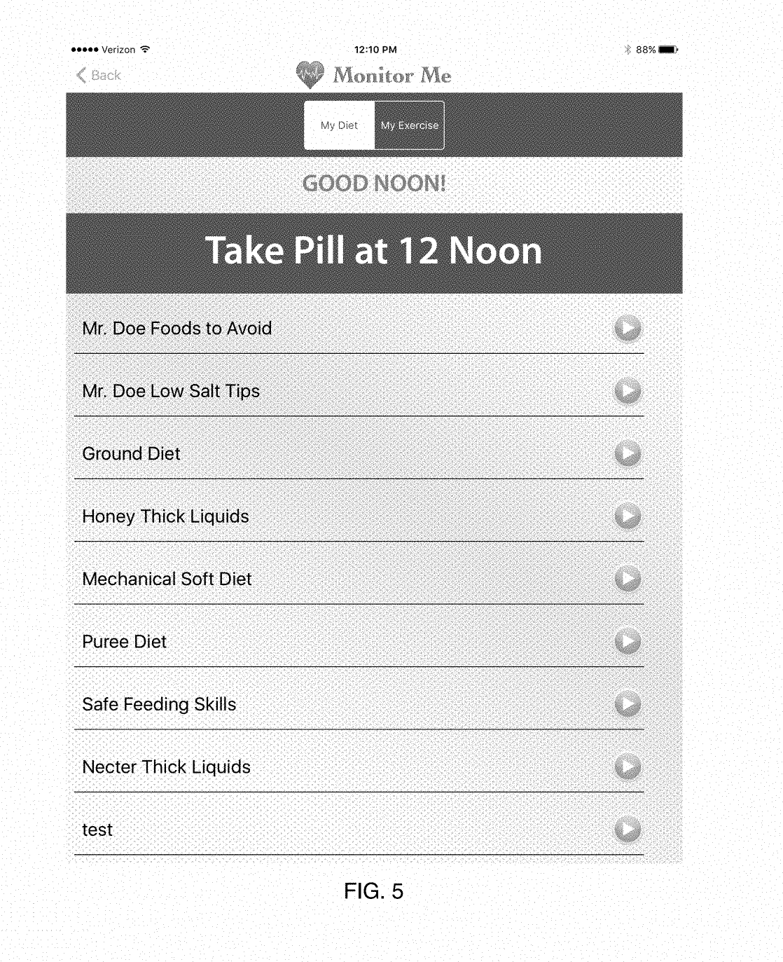

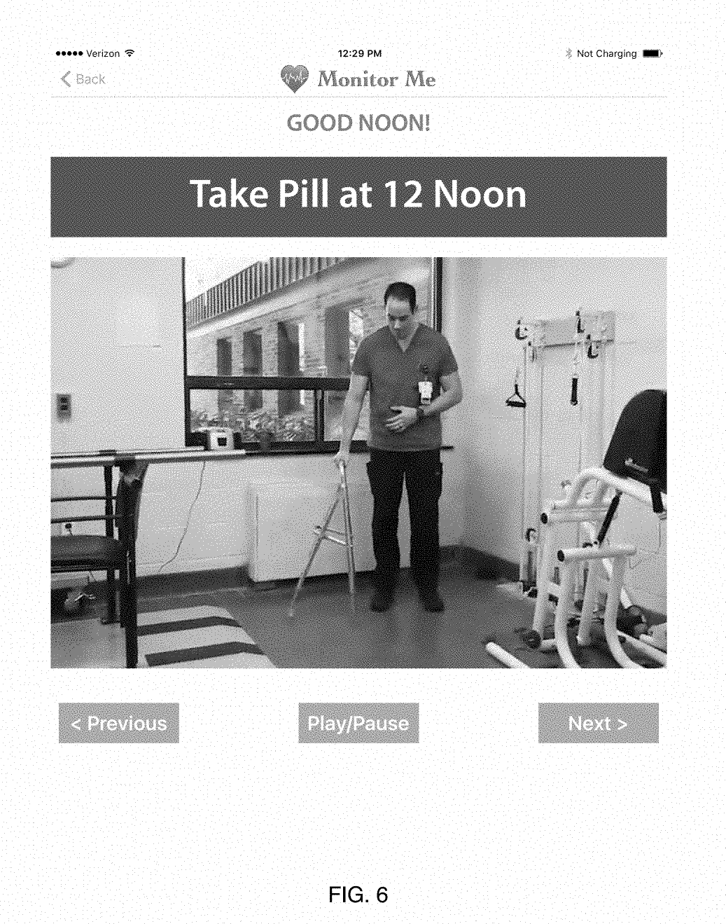

As shown on the images from the portable computing device in FIGS. 5-6, a “Take Pill at 12 Noon” reminder is present throughout. This feature reminds the patient of their medication schedule regardless of the section of the device that they are utilizing. More specific medication information may be obtained in the “My Pills” section show in FIG. 4.

The table in FIG. 6 shows the patient information about the various medications prescribed to the patient, and lists the dosage, form and frequency. Each medication may also have a brief explanation and an image for size/color.

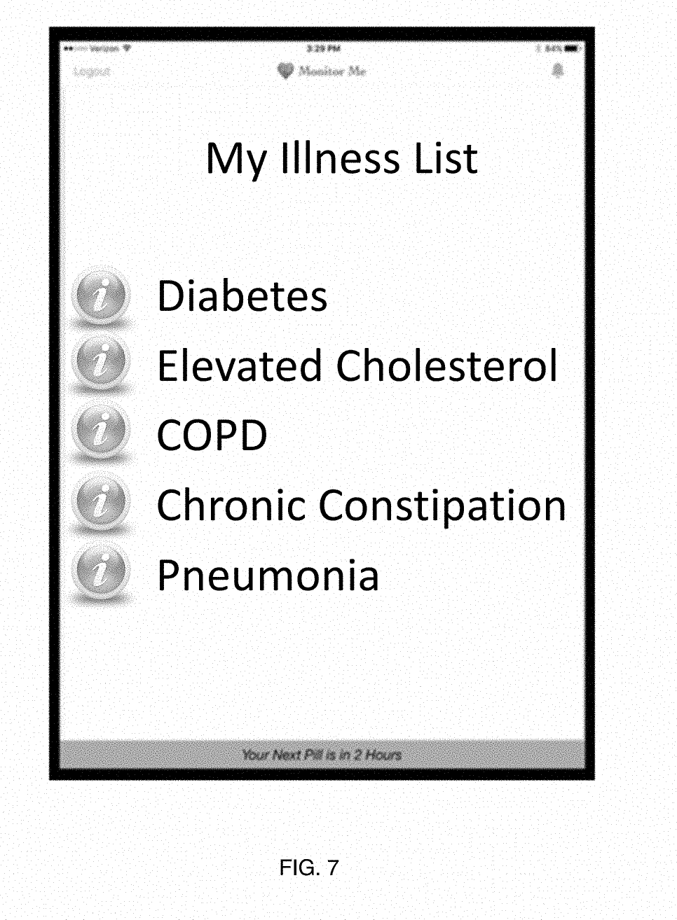

A My Illness, shown in FIG. 7, feature can provide patient-specific illness information that can include specific information on the patient's condition that may be presented as videos or in documentary. In embodiments, the My Illness function may list patient illnesses and information regarding each specific disease process through integration with evidenced based support resources. Patients can learn more about a specific disease by selecting the information icon.

A diet function may provide patient-specific diet information to assist the patient in making appropriate choices for their condition. A portal may be provided where the patient may view videos specific to diets related to their illnesses/conditions, and access other information curated by the healthcare provider.

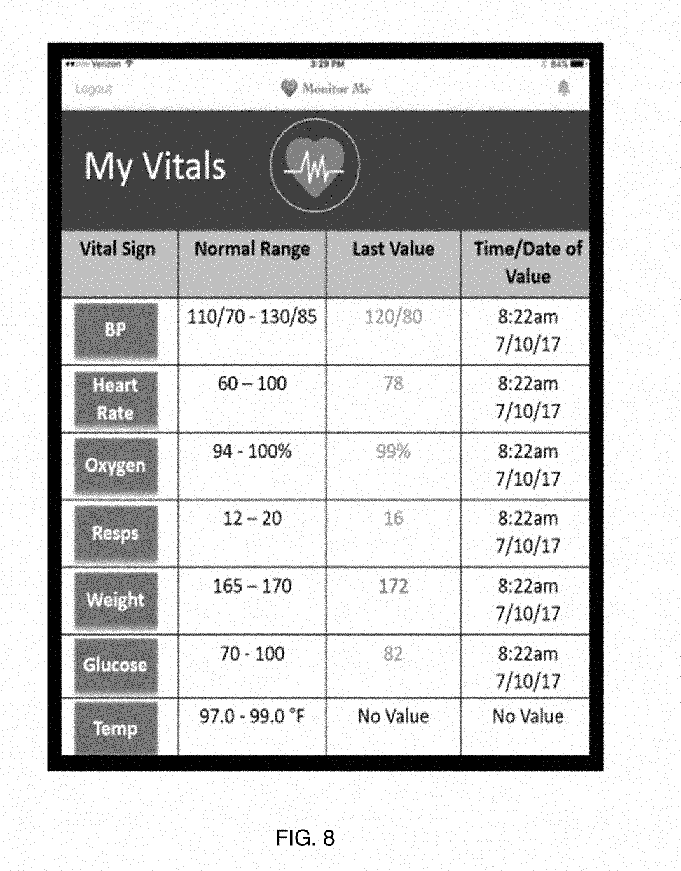

FIG. 8 shows an interface wherein vital signs may be recorded over a period of time, and for multiple parameters. The vitals section displays information gathered from the various sensor devices such as the telemetry monitor, pulse monitor, and the like.

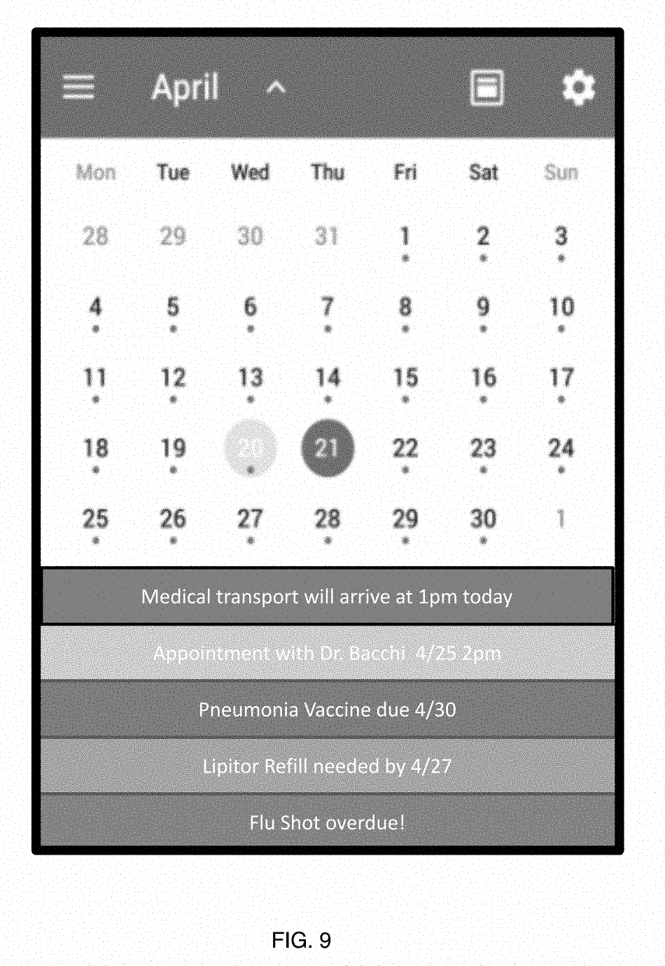

An appointments section may also remind the patient of upcoming visits to healthcare providers, and the purpose of the visit, as shown in FIG. 9. As with the medication reminder, reminders may appear on all screens for upcoming appointments.

The foregoing examples are not intended to be limiting and any number of additional treatment dimensions may be integrated into a patient device integrated with the system. For example, an exercise portal may display patient-specific exercise information and provide information on exercises relevant to the patient's condition to help maintain their health. A preventative care function may display patient preventative care that is upcoming or already managed. Immunizations, flu/pneumonia shots, dental visits, eye exams, and the like are examples of what might be covered in this function. In embodiments, patients can request flu/pneumonia shots and the nurse would get an email about scheduling these care requests.

Lastly, a 911 or emergency function summons emergency services such as police, fire, or ambulance.

In embodiments, the system may enable the transmission of real-time time hemodynamic data—including six-lead EKG, heart rate, respiration, blood oxygen level, blood pressure, cardiac output, cardiac index, stroke volume, and systemic vascular resistance. By sending data from wearable FDA-approved devices to a care management unit (CMU), a new level of oversight is able to keep patients in optimal wellness at home. Hospital visits are avoided and preventative care is timely administered

Cognitive computers analyze data in real-time to provide predictive insight and catch life threatening incidents prior to them happening. A CMU staffed by trained healthcare personnel supplements the analysis and is present to evaluate and monitor developing health issues in real time.

In addition to acute care, the present invention enables customized assistance such as a personalized diet from a registered dietician, personalized exercise programs, and personalized information to better inform patients about their condition and care options.

In more serious situations, immediate contact with nurses and doctors is facilitated, and critical intervention can be summoned.

On a daily basis, the present invention can educate the user and caregiver and/or family member on when medication must be taken, what exercises should be performed, what diet should be followed, pertinent health tips, appointment times, and when there is a change in conditions. As a result, quality of life is improved, lives may be saved, and costs drastically reduced.

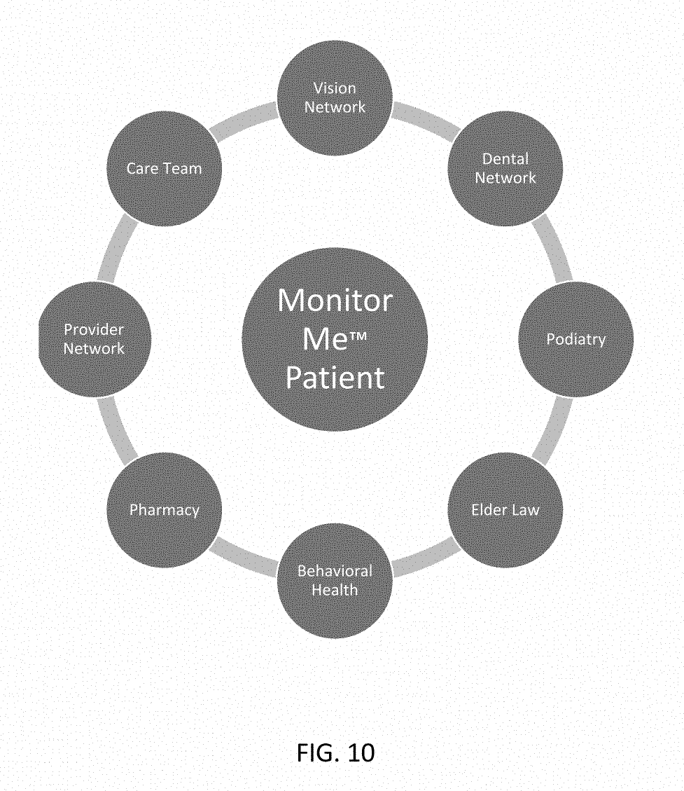

A holistic overview of the system is show in in FIG. 10. A patient participating in the system of the present invention may be provided with not only real-time healthcare monitoring, but have access to a suite of patient services across multiple treatment dimensions. Integrations may be provided with patient healthcare records, caregiver contact information, provider network information (e.g., vision, dental and behavioral health), pharmacy network facilitating refills and medication deliveries, and non-medical services such as elder law.

In the above description, numerous details are set forth. It will be apparent, however, to one of ordinary skill in the art having the benefit of this disclosure, that embodiments of the invention may be practiced without these specific details.

Unless specifically stated otherwise as apparent from the above discussion, it is appreciated that throughout the description, discussions utilizing terms such as “searching,” “determining,” “calculating,” “obtaining,” “transmitting,” “receiving,” “querying,” “processing,” or the like, refer to the actions and processes of a computing device, or similar electronic computing device, that manipulates and transforms data represented as physical (e.g., electronic) quantities within the computing device's registers and memories into other data similarly represented as physical quantities within the computing device memories or registers or other such information storage, transmission or display devices.

Embodiments of the invention also relate to an apparatus for performing the operations herein. This apparatus may be specially constructed for the required purposes, or it may comprise a general-purpose computer selectively activated or reconfigured by a computer program stored in the computer. Such a computer program may be stored in a non-transitory computer readable storage medium, such as, but not limited to, any type of disk including floppy disks, optical disks, CD-ROMs and magnetic-optical disks, read-only memories (ROMs), random access memories (RAMs), EPROMs, EEPROMs, magnetic or optical cards, flash memory, or any type of media suitable for storing electronic instructions.

The words “example” or “exemplary” are used herein to mean serving as an example, instance, or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application should generally be construed to mean “one or more” unless specified otherwise or clear from context to be directed to a singular form. Moreover, use of the term “an embodiment” or “one embodiment” or “an implementation” or “one implementation” throughout is not intended to mean the same embodiment or implementation unless described as such.

Any algorithms and displays presented herein are not inherently related to any particular computer or other apparatus. Various general-purpose systems may be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the invention as described herein.

The above description sets forth numerous specific details such as examples of specific systems, components, methods and so forth, in order to provide a good understanding of several embodiments of the present invention. It will be apparent to one skilled in the art, however, that at least some embodiments of the present invention may be practiced without these specific details. In other instances, well-known components or methods are not described in detail or are presented in simple block diagram format in order to avoid unnecessarily obscuring the present invention. Thus, the specific details set forth above are merely exemplary. Particular implementations may vary from these exemplary details and still be contemplated to be within the scope of the present invention.

It will be understood that there are numerous modifications of the illustrated embodiments described above which will be readily apparent to one skilled in the art, including any combinations of features disclosed herein that are individually disclosed or claimed herein, explicitly including additional combinations of such features. These modifications and/or combinations fall within the art to which this invention relates and are intended to be within the scope of the claims, which follow. It is noted, as is conventional, the use of a singular element in a claim is intended to cover one or more of such an element.

We claim:

1. A method for evaluating patient health and reducing patient relapse after treatment comprising:
establishing a communication channel with at least one patient monitoring device that has been configured to measure a physiological characteristic of a patient;
establishing a second communication channel with a remote care management unit;
receiving a first patient data object describing the physiological characteristic of a patient, evaluating the first patient data object against predetermined criteria, and causing an alert condition when the first patient data object is outside the predetermined criteria; and
receiving the alert condition at the remote care management unit and facilitating a communication link between the portable communication device and the remote care management unit such that the need for further intervention can be determined.

2. The method of claim 1 in which the portable computing device is one of: a smartphone computing device, a tablet computing device, and a laptop computing device.

3. The method of claim 1 in which the portable computing device is a smart television device or a streaming device.

4. The method of claim 1 in which the patient monitoring device is a telemetry device used to monitor heart rate and rhythm.

5. The method of claim 4 in which the telemetry device is connected to the patient using a disposable patch.

6. The method of claim 7 in which the hemodynamic data includes electrocardiograms data from a monitoring device having at least six leads.

7. The method of claim 1 in which remote care management unit receives hemodynamic data in real time over the second communication channel.

8. The method of claim 1 in which the remote care monitoring unit is a central facility staffed by healthcare professionals to monitor patient wellness.

9. The method of claim 1 in which the remote care monitoring unit comprises at least one cognitive computing device configured to analyze all incoming patient data objects data to identify potential critical care situations.

10. The method of claim 1 in which the portable computing device is configured to provide interactive access to at least one of: a patient medication portal, a diet and nutrition portal, a physical therapy portal, an illness portal, a vital signs portal, and an appointments portal.

11. A non-transitory computer readable storage medium storing one or more sequences of instructions, when executed by one or more processors, to cause the one or more processors to perform a set of operations comprising:
establishing, on a portable computing device, a communication channel with at least one patient monitoring device that has been configured to measure a physiological characteristic of a patient;
establishing a second communication channel with a remote care management unit;
receiving a first patient data object describing the physiological characteristic of a patient, and evaluating the first patient data object against predetermined criteria, and causing an alert condition when the first patient data object is outside the predetermined criteria; and
receiving the alert condition at the remote care management unit and facilitating a communication link between the portable communication device and the remote care management unit such that the need for further intervention can be determined.

12. The non-transitory computer readable storage medium of claim 11 in which the portable computing device is one of: a smartphone computing device, a tablet computing device, and a laptop computing device.

13. The non-transitory computer readable storage medium of claim 11 in which the portable computing device is a smart television device or a streaming device.

14. The non-transitory computer readable storage medium of claim 11 in which the patient monitoring device is a telemetry device used to monitor heart rate and rhythm.

15. The non-transitory computer readable storage medium of claim 14 in which the telemetry device is connected to the patient using a disposable patch.

16. The non-transitory computer readable storage medium of claim 11 in which the remote care management unit receives hemodynamic data in real time over the second communication channel.

17. The non-transitory computer readable storage medium of claim 16 in which the hemodynamic data includes electrocardiograms data from a monitoring device having at least six leads.

18. The non-transitory computer readable storage medium of claim 11 in which the remote care monitoring unit is a central facility staffed by healthcare professionals to monitor patient wellness.

19. The non-transitory computer readable storage medium of claim 11 in which the remote care monitoring unit comprises at least one cognitive computing device configured to analyze all incoming patient data objects data to identify potential critical care situations.

20. The non-transitory computer readable storage medium of claim 11 in which the portable computing device is configured to provide interactive access to at least one of: a patient medication portal, a diet and nutrition portal, a physical therapy portal, an illness portal, a vital signs portal, and an appointments portal.
